(19) United States
(12) Patent Application Publication
(10) Pub. No.: US 2026/0171250 A1
(43) Pub. Date: Jun. 18, 2026

(54) DENTAL SYSTEM AND SOFTWARE FOR MAKING REMOVABLE DENTAL PROSTHETICS

(71) Applicant: Olugbenga Abimbola Akinsanya, Waterville, ME (US)

(72) Inventor: Olugbenga Abimbola Akinsanya, Waterville, ME (US)

(21) Appl. No.: 19/530,547

(22) Filed: Feb. 5, 2026

Related U.S. Application Data
(62) Division of application No. 18/929,974, filed on Oct. 29, 2024.
(60) Provisional application No. 63/605,864, filed on Dec. 4, 2023.

(51) Int. Cl. G16H 50/20; A61B 5/107; A61C 13/00; G16H 10/60; G16H 20/40

(52) U.S. Cl. CPC G16H 50/20; A61B 5/1072; A61C 13/0004; A61C 13/0019; G16H 10/60; G16H 20/40

(57) ABSTRACT

The present application discloses a dental system and software configured to obtain facial measurement data associated with a patient and create a patient removable dental prothesis based on the facial measurement data by using Artificial Intelligence (AI).

### CROSS-REFERENCE TO RELATED APPLICATION

This application is a divisional of U.S. Non-Provisional patent application Ser. No. 18/929,974, filed Oct. 29, 2024, which claims the benefit of priority under 35 U.S.C. § 119(e) to U.S. Provisional Patent Application No. 63/605,864, filed Dec. 4, 2023, the entire contents of each of which are incorporated herein by reference.

### BACKGROUND OF THE INVENTION

The present invention relates generally to a dental system and software for making dentures, and specifically to a dental system and software for making removable dental prosthetics (including complete and partial dentures) from radiographs, facial scans, pictures, videos, and initial impressions, models or intraoral scans and other unique anatomical, behavior and psychological features of the individual patient obtained at the initial patient visit.

Currently, dental care providers have to see patients for multiple visits for multiple procedures and take numerous intraoral measurements to make complete or partial dentures. Such multiple visits create inconvenience to the patients and the dental care providers. Further, this procedure of making the dentures is cumbersome, time consuming, repetitive and prone to a lot of errors. Existing systems to make the dentures take too much time and are prone to errors leading to inaccuracy of the removable dental prosthesis (complete or partial dentures).

Therefore, a system and software is required to efficiently make removable dental prosthesis—complete or partial dentures.

### SUMMARY OF THE INVENTION

The following presents a simplified summary of the present disclosure in a simplified form as a prelude to the more detailed description that is presented herein.

In accordance with preferred embodiments of the present invention, there is provided an automated dental system for making a removable prosthetic appliance with artificial intelligence using data associated with a patient, obtained by a dental care provided or anyone so designated by the dental care provider during the initial visit to the dental provider.

The dental system, as disclosed in the present disclosure, uses computer software data from patient's own oro-facial biology and extended dental golden proportions to make dentures, by-passing numerous clinical and technical steps. The dental system is different from conventional systems for making dentures because the dental system and software does not require any work or effort by a human clinician and technician to fabricate denture or removable dental prosthesis. The dental system uses Artificial Intelligence (AI) based algorithms to create the dentures based on the input data about the patient's oro-facial anatomy.

These and other features, aspects and advantages of the present invention will become better understood with reference to the following description and claims.

### BRIEF DESCRIPTION OF THE DRAWINGS

Illustrative embodiments of the present invention are described herein with reference to the accompanying drawings, in which:

FIGS. 1A and 1B depicts a flow chart relating to the use of artificial intelligence to replace missing teeth with removable dental appliances using golden proportional measurement of existing relational structures of the orofacial complex.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

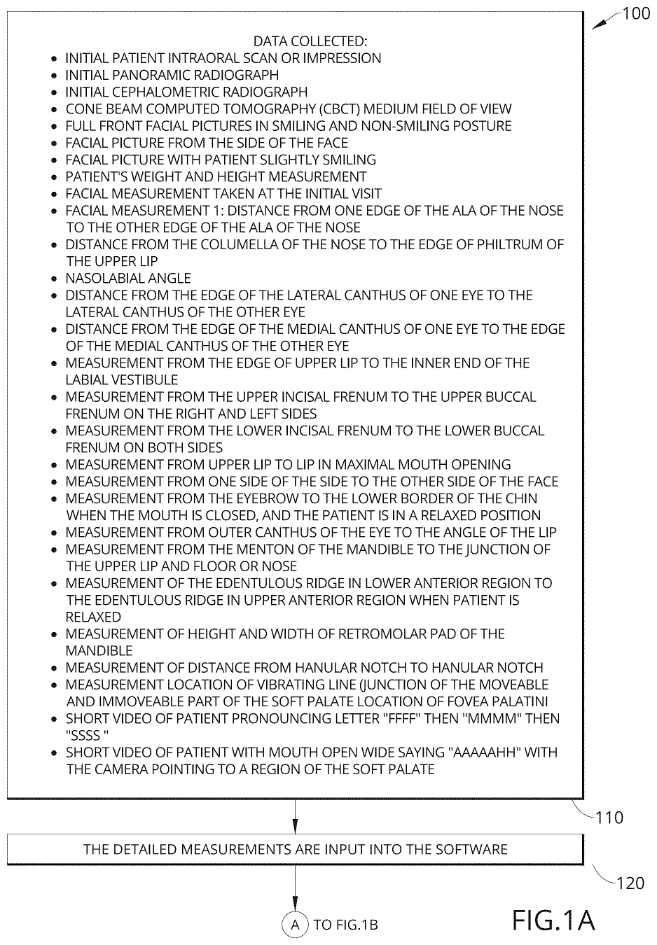

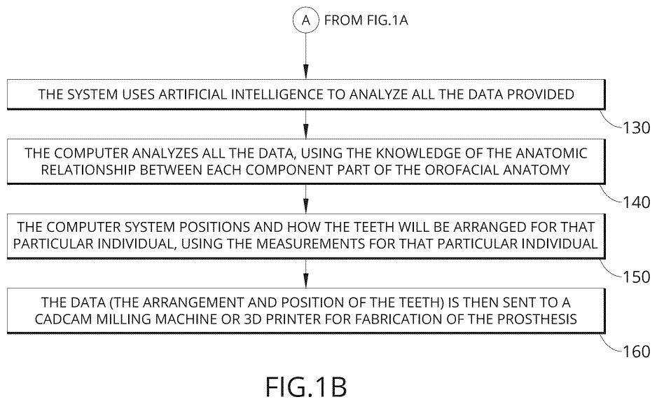

FIG. 1 depicts a block diagram of a dental system 100 (or system 100) in accordance with preferred embodiments of the present invention. The system 100 uses artificial intelligence (AI) to create a complete removable dental prosthesis from a patient's own oro-facial anatomy captured during the initial dental visit. This data is acquired by most dentists during a dental new patient visit (and hence is easily available), but it is never used or there is currently no computer software/system that computes all these data with an algorithm to finish the denture process. The initial patient data used by the system 100 includes panoramic radiograph, cephalometric radiograph, oro-facial CT scan, facial scan, intraoral impression or intraoral scan, extraoral and intraoral photographs of the patient taken during the initial exam visit, historical smiling photographs (if available) of the patient when he/she had her natural teeth, and/or the like.

The system 100 uses artificial intelligence and computer software to by-pass many and/or all of the steps currently performed by clinicians and technicians in fabricating dentures. The system 100 only uses the initial data obtained from the patient's orofacial anatomy, and hence significantly enhances patient and dentist's convenience and produce a more accurate removable prothesis.

In an exemplary aspect, the patient data 110 used by the system 100 to make the denture includes, but is not limited to, initial patient intraoral scan or impression, initial panoramic radiograph, initial cephalometric radiograph, Cone Beam Computed Tomography (CBCT) medium field of view, full front facial pictures in smiling and non-smiling posture, facial picture from the side of the face, facial picture with patient slightly smiling, patient's weight and height measurement, patient's facial measurement taken at the initial visit (though this measurements can be easily obtained from good patient pictures, but it is better to take the measurements separately), and/or the like.

The facial measurement may include, for example, distance from one edge of ALA of the nose to the other edge of ALA of the nose, distance from the columella of the nose to the edge of philtrum of the upper lip, nasoliabial angle, distance from edge of lateral canthus of one eye to the lateral canthus of the other eye, distance from edge of medial canthus of one eye to the edge of medial canthus of the other eye, measurement from the edge of upper lip to the inner end of the labial vestibule, measurement from upper incisal frenum to the upper buccal frenum on right and left sides, measurement from lower incisal frenum to the lower buccal frenum on both sides, measurement from upper lip to lower lip in maximal mouth opening, measurement from one side of the side to the other side of the face, measurement from the eye brow to the lower border of the chin (mental region) when the mouth is closed and patient is in a relaxed position, measurement from the menton of the mandible to the junction of the floor of the nose and upper lip, measurement from the lateral canthus of the eye to the angle of the mandible, and/or the like.

A person ordinarily skilled in the art may appreciate all the measurements obtained for the patient (described above) have a well-defined proportional relationship in every normal human being that society takes into consideration when an individual is regarded as beautiful or handsome. Wherever any of these measurements is out of proportion, society currently prescribes different oro-facial procedures such as plastic surgery, maxillofacial surgery, orthodontics procedure, cosmetic dentistry procedure, etc., to correct the part of the measurement that is out of place. As an example, the lower anterior teeth relationship with the mandibular plane angle should be at 90 degrees, the edge of the lower teeth should be at the level of the mouth commissure, the position of the upper anterior teeth determines the nasolabial angle which, depending on the patient's ethnic makeup should be slightly acute (e.g., for Africans), 90 degrees or obtuse (e.g., for Caucasian). As further examples, the ala or the nose has a special relationship with the position of the maxillary canines, the central incisors are located on either side of the labial frenum at a specific distance and angle from the nasopalatine papilla, and at a specific angle from the maxillary plane angle, the buccal frenum is located in relation to the premolar teeth, and/or the like.

All the patient-specific measurements obtained by the system 100 at the initial dental visit of a patient have specific relationship with the teeth position in the mouth that is unique for that patient. The system 100 uses artificial intelligence to position the teeth in an ideal position for that particular patient using the patient-specific data input for that particular patient with an algorithm that is created in the computer software installed in the system 100. Depending on the jurisdiction, all the data can be obtained by trained dental auxiliaries working under supervision of the dentist (general or direct supervision). The data 110 is provided to the computer software (step 120) installed in the system 100 and the denture process is made based on the algorithm established using the proportional relationship of each part of the orofacial anatomy in a normal human being.

Once detailed patient measurements are input into the computer software installed in the system 100 (step 120), the system 100 uses artificial intelligence to analyze all the data provided (step 130). The patient may be missing all or some of the teeth. The system 100 analyzes all the data, using the knowledge of the anatomic relationship between each component/part of the orofacial anatomy, and positions the teeth for that particular patient using the measurements for that particular patient (steps 140 and 150). The data (the arrangement and position of the teeth) is then sent to a CADCAM milling machine or a 3D printer for fabrication of the prosthesis (step 160). This step completely bypasses the multiple human-clinical and technical steps that are currently done by the clinicians and technicians.

In some aspects, if some parts of the orofacial complex are missing but teeth are present, the reverse can be used by the system 100 to determine the location and replacement of the missing parts of the orofacial complex using the same proportional relationships of different parts of the orofacial complex.

In additional aspects, the system 100 can also be used to reconstruct face in cases of a missing person, forensic odontology, orthodontics, orthognathic surgery, restoration of missing parts of orofacial complex as a congenital malformation, burns, accidents, diseases, plastic and cosmetic surgery, dentistry, etc.

The mandibular molar teeth's buccal cusp tip (1st and 2nd molars), premolar and anterior teeth will be at the level of ½ to ⅔rd the height of retromolar pad with all mandibular teeth set to a flat or slight curve of spee. The incisal edge of anterior teeth and the premolars and molars all form the mandibular occlusal plane will be at the height of ½ to ⅔rd of the mandibular retromolar pad and when projected posteriorly to head of mandibular condyle.

The lingual cusp tips should be slightly (about 0.5 mm) lower than the buccal cusp tips to create a gentle curve of wilson. The anterior teeth incisal edge will be at around the level of an imaginary line drawn from the angle of the lip on one side to the angle of lip on the other side. The mandibular arch length will be measured from the space in front of one retromolar pad to the space in front of the other retromolar pad. This arch length will determine the dimensions (mesio-distal width of the mandibular teeth. The location of the mental foramen observable from 3D CBCT or panoramic radiograph will determine location of the premolars—68 percent of the people, the mental foramen is located below the apex of mandibular second premolar and 17 percent, the mental foramen is located between first and second premolar apices while in 11% of population it is located below the apex of mandibular first premolar.

The posterior extent of the mandibular teeth should terminate around the region where the residual ridge begins its ascend vertically to the retromolar pad. The arrangement of teeth should conform to the CURVE OF MONSON. The posterior extent of the maxillary denture is located at the posterior vibrating line-an imaginary line that marks the junction between the moveable and immovable part of the soft palatal (This information is obtained from the short video of patient with mouth wide opened saying ‘Aaaaah’). This area is where the posterior palatal seal is located. The seal is created by indentation of the posterior edge of the upper denture between 0.5-1 mm and extends from one hanular notch to the other hanular notch. a few mm anterior to the fovea palatini. Historically, dentists determine this line by telling the patient to say a prolonged ‘Aaaah’ and a marker is used to mark this line and transferred to the denture base plate. However, with my invention, this clinical procedure step can be avoided. Based on information obtained from clinical picture and short recorded 30 second video of clinician during the initial visit telling patient to say the word ‘Aaaaah’ and 2D and 3D CDCT radiographs, the step of having the clinician performing this procedure during a separate visit can be eliminated and the Artificial intelligence can easily and more accurately locate this area of the posterior extent of the maxillary denture and create the postdam area.

### Vertical Dimension of Occlusion and Free Way Space:

The concept of Vertical dimension of occlusion of denture when the set up of teeth in occlusion and in maximum intercuspation, the measurement from menton to the junction of the nose and upper lip (unstable reference point for vertical dimension of occlusion) should be equal to measurement from the distance from the outer canthus of the eye to the angle of the lip. This is a stable reference point for Artificial intelligence to use for determination of the VDO of the new denture without any additional unnecessary clinical or laboratory step by dentists or technicians as its currently being carried out.

The unstable reference point: Measurement of distance from menton of the mandible to lip/nose junction is not a consistent reference point. The obtained measurement depends on whether there is any appliance in the mouth or if patient has teeth or not. However, in an ideal, facial appearance that the society considers beautiful or attractive, with normal arrangement of teeth or dentures existing in the mouth, the measurement from the mandibular menton to nose/lip junction (unstable reference point) should be equal to measurement from the outer canthus of the eye to the angle of the mouth (stable reference point).

In the process of constructing a removable dental prosthesis using the system described here, the artificial intelligence can easily determine what the distance from mandibular menton to the nose/lip junction should be from the figure obtained of the stable reference point (measurement from outer canthus of eye to the angle of lip). The computer system can easily determine what vertical height of the teeth in the removable prosthesis should be set to arrive at the figure for the normal VDO after allowing for thickness of the denture base plate of between 1.0 mm to 2.5 mm and soft tissue thickness overlying the mandibular and maxillary bone. Information about soft tissue thickness is obtainable from the CBCT 3D imaging.

An additional method for the software to arrive at correct Vertical Dimension of Occlusion is by allowing 19 mm +/−1 mm for the maxillary and mandibular anterior denture teeth measured from the gingival crest of anterior denture teeth to the gingival crest of mandibular denture teeth, when the teeth are in occlusion at normal Overbite/overjet of 1 mm. This is the so called Shimbashi measurement.

Teeth setup using the proportional measurements, upper and lower teeth are brought together in maximum intercuspation using the stable measurement as a guide, and the space between the teeth and the soft tissue covering the madibular and maxillary bone are filled with denture base plate.

The accuracy of the Vertical Dimension of Occlusion, using the patient's existing anatomy and golden proportions from all the extensive data collected at the initial appointment by artificial intelligence, will allow for the freeway space. The free way space is crucial to enable the patient to use his/her removable dental prosthesis comfortably without any occlusal interferences.

This invention relies on data collection at the initial visit. That is all that is needed. Dentists already use the first appointment visit for data collection about their patients anyway. The data collection can be done directly by the Dentist or whoever the dentist delegates the task to, under general or direct supervision depending on the dental practice law in the jurisdiction where the service is being performed (general supervision meaning the dentist does NOT have to be in attendance during this visit OR direct supervision meaning the dentist has to be in attendance during the data collection patient visit direct action by dentist meaning the dentist has to perform the data collection).

Data collection should take a trained provider or delegated person about 30 minutes. The data collection is very similar to records taken in orthodontists offices during the initial orthodontic visit.

Once all the collected data is input into the system, the removable prosthesis can be generated within 30 minutes without any additional steps. Depending on the state or jurisdiction of service location, a patient may be asked to return to office for a trial fit of the removable prosthesis before a final delivery appointment.

The purpose of the trial fit appointment is for the patient to see, feel and try the denture before final delivery. The denture that is provided to the patient during the try-in visit should be the same or very similar to final prosthesis.

Except as may be expressly otherwise indicated, the article “a” or “an” if and as used herein is not intended to limit, and should not be construed as limiting, the description or a claim to a single element to which the article refers. Rather, the article “a” or “an” if and as used herein is intended to cover one or more such elements, unless the text expressly indicates otherwise.

It should be understood, of course, that the foregoing relates to exemplary embodiments of the invention and that modifications may be made without departing from the spirit and scope of the invention as set forth in the following claims.

What is claimed is:

1. A method of making a removable dental prosthesis comprising:
gathering facial measurement data of a patient during an initial visit, the facial measurement data comprising panoramic radiograph, cephalometric radiograph, oro-facial CT scan, facial scan, intraoral impression or intraoral scan data or photographs;
using artificial intelligence (AI) to create a complete removable dental prosthesis from said data.

2. The method of claim 1, wherein the facial measurement data further comprises at least one of: patient weight, patient height, facial measurements, historical photographs of the patient with natural teeth, extraoral photographs, or intraoral photographs.

3. The method of claim 1, wherein using artificial intelligence comprises applying golden proportional relationships between different parts of an orofacial complex to determine tooth placement and dimensions.

4. The method of claim 1, further comprising determining a vertical dimension of occlusion by measuring a distance from an outer canthus of an eye to an angle of a lip as a stable reference point.

5. The method of claim 1, wherein creating the complete removable dental prosthesis comprises determining a posterior vibrating line location from video data of the patient saying a prolonged sound captured during the initial visit.
